(19) United States
(12) Patent Application Publication
(10) Pub. No.: US 2010/0129889 A1
(43) Pub. Date: May 27, 2010

(54) AFFINITY SEPARATION METHODS AND SYSTEMS

(75) Inventor: Jens Sommer-Knudsen, New South Wales (AU)

Correspondence Address: MORGAN LEWIS & BOCKIUS LLP, 1111 PENNSYLVANIA AVENUE NW, WASHINGTON, DC, 20004, US

(73) Assignee: INNOVATIVE PURIFICATION TECHNOLOGIES PTY LTD, Everleigh, NSW (AU)

(21) Appl. No.: 12/439,968

(22) PCT Filed: Aug. 31, 2007
(86) PCT No.: PCT/AU2007/001272

§ 371 Date: Jan. 8, 2010

(30) Foreign Application Priority Data
Sep. 5, 2006 (AU) 2006904859

(51) Int. Cl. C07K 1/22; C07H 21/00; C08G 63/91; C12N 9/00; C07H 1/06; C07K 9/00; C08H 1/00; C12N 7/02; C12N 1/00

(52) U.S. Cl. 435/183; 435/239; 435/243; 525/54.1; 530/344; 530/391.1; 530/395; 530/408; 530/413; 536/25.4; 536/123.1; 536/127; 435/261; 435/325

(57) ABSTRACT

An affinity matrix comprising a base matrix containing biotin; and a fusion protein attached to the base matrix, wherein the fusion protein contains a matrix binding element capable of binding to the base matrix via biotin and a target binding element capable of binding, or being bound by, at least one target component.

### TECHNICAL FIELD

The present invention relates to matrices, systems, methods and kits suitable for affinity separations.

### BACKGROUND OF THE INVENTION

Affinity separation is usually performed using a hydrophilic matrix, to which an affinity ligand is covalently linked. Typically this matrix consists of small beads or membranes to which proteins or other affinity ligands have been covalently bound. Proteins bound to the matrix typically must undergo an extensive purification process prior to being bound to the matrix, and the protein must subsequently be covalently coupled to the matrix under gentle conditions, which do not impinge on the biological activity of the molecule. Thus, affinity separation matrices containing immobilised proteins tend to be very expensive.

The present invention is predicated on the unexpected and surprising finding that fusion proteins where at least one part of the fusion protein has a strong affinity to a base matrix, and at least one other part of the fusion protein has an activity or function suitable for interaction with other biological molecules, can be used to provide an affinity matrix suitable for affinity separations as an alternative to affinity matrices containing covalently bound purified protein or peptide ligands.

### SUMMARY OF THE INVENTION

In a first aspect, the present invention provides an affinity matrix for binding a target component comprising:

(a) a base matrix containing biotin; and

(b) a fusion protein attached to the base matrix, wherein the fusion protein contains a matrix binding element capable of binding to the base matrix via biotin and a target binding element capable of binding, or being bound by, at least one target component.

The base matrix maybe soluble or insoluble. Preferably, the base matrix is a particulate matrix such as beads made from cellulose, agarose, dextrane, acrylamide, polysulfone, polyamide, silica, or other suitable materials or combinations thereof typically used as matrices for separation of biological macromolecules.

Alternatively the base matrix may be a continuous matrix such as a membrane made from cellulose, acrylamide, polysulfone, polyamide, polyvinylidene difluoride (PVDF), polyvinyl chloride (PVC), polypropylene, polyester, silica or any other suitable materials or combinations thereof.

The base matrix is preferably derivatised with biotin or derivatives thereof.

The affinity matrix may comprise a plurality of fusion proteins.

In one preferred embodiment, the matrix binding element is avidin or derivatives and variations thereof.

Preferably, the matrix binding element is streptavidin.

The target binding element maybe selected from immunoglobulin binding agents such as protein A, protein G or protein L, an antibody binding domain, a single chain antibody, an enzyme, an inhibitor, an antigenic determinant, an epitope, a binding site, a lectin, a cellulose binding protein, a polyhistidine, an oligohistidine, a receptor, a hormone, a signalling molecule, a polypeptide with specific or group specific binding capabilities, or combinations thereof.

In one embodiment, the target binding element is capable of binding an immunoglobulin. Preferably, the target binding element is protein A, protein G, or protein L. More preferably, the target binding element is protein A.

The fusion protein may be generated by recombinant DNA technology.

The fusion protein may be attached to the base matrix by mixing the base matrix with material such as an extract, supernatant, secretion, or lysate containing the fusion protein. Alternatively the material can be passed through a bed, a column, or a sheet of the base matrix. The at least one part of the fusion protein with strong affinity for the base matrix will bind to the base matrix, thus providing an affinity matrix with at least one remaining part of the fusion protein being able to bind, or be bound by, a target component. Any undesired components of the material containing the fusion protein can be washed away from the affinity matrix by the use of suitable solutions such as buffers, that preferably do not cause substantial release of the fusion protein from the base matrix. The base matrix may be derivatised with biotin and the part of the fusion protein with strong affinity for the base matrix may be streptavidin or variations thereof. Biotin can be covalently bound to a base matrix through a variety of chemical reactions known to those skilled in the art.

In a second aspect, the present invention provides a method of making an affinity matrix comprising:
(a) forming a base matrix by derivatising a matrix with biotin;
(b) providing a fusion protein, wherein the fusion protein contains a matrix binding element capable of binding to the base matrix via biotin and a target binding element capable of binding, or being bound by, at least one target component; and
(c) attaching the fusion protein to the base matrix via biotin.

In a third aspect, the present invention provides a method for separating at least one target component from a mixture comprising:

(a) providing a mixture containing a target component to an affinity matrix according to the first aspect of the present invention; and

(b) allowing a target component in the mixture to bind to the affinity matrix via the target binding element.

Preferably, the method further comprises:

(c) recovering the target component from the matrix.

The method according to the present invention can be used to enrich at least one desired component within a mixture by separating at least one undesired target component from the mixture.

When a mixture is contacted with the affinity matrix the target component selectively binds, or is selectively bound by, at least one part of the fusion protein. The mixture may comprise any suspension, dispersion, solution or combination thereof of any biological extracts or derivatives thereof. For example, the mixture may include blood, blood plasma, blood serum, blood derived precipitates or supernatants, animal extracts or secretions, milk, colostrum, whey or any other milk derived product or fraction thereof, fermentation broths, liquids or fractions thereof, cell lysates, cell culture supernatants, cell extracts, cell suspensions, viral cultures or lysates, plant extracts or fractions thereof.

The target component may comprise a protein, a peptide, a polypeptide, an immunoglobulin, biotin, an inhibitor, a co-factor, a substrate, an enzyme, a receptor, a monosaccharide, an oligosaccharide, a polysaccharide, a glycoprotein, a lipid, a nucleic acid, a cell or fragment thereof, a cell extract, an organelle, a virus, a biological extract, a hormone, a serum protein, a milk protein, a milk-derived product, blood, serum, plasma, a fermentation product a macromolecule or any other molecule or any combination or fraction thereof. The biological extract may be derived from any plant, animal, microorganism or protista.

The target component may be a desired component for subsequent use or an undesired component that needs to be removed from a desired component or components in a sample. The target component may be an undesired contaminant.

The target component may be recovered from the affinity matrix to which the target component is bound. The recovery of the target component may be via at least one elution step wherein the binding of the target component to the fusion protein is weakened, disrupted, broken or competitively substituted.

The affinity matrix according to the present invention may be used to obtain a desired component or an undesired component in a sample.

In a fourth aspect, the present invention provides use of the affinity matrix according to the first aspect of the present invention to separate or enrich at least one target component.

In a fifth aspect, the present invention provides a kit for affinity separation comprising:

(a) an affinity matrix according to the first aspect of the present invention; and

(b) diluent and/or eluent for carrying out an affinity separation using the matrix.

In a preferred form, the kit further contains instructions to carry out an affinity separation.

Throughout this specification, unless the context requires otherwise, the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed in Australia prior to development of the present invention.

In order that the present invention may be more clearly understood, preferred embodiments will be described with reference to the following drawings and examples.

### BRIEF DESCRIPTION OF THE DRAWINGS

FIG. 1 shows nucleic acid and protein sequence of streptavidin-zz domain used as an example of a suitable matrix binding element for the fusion protein. SEQ ID NO: 1 is nucleotide sequence of upper strand of DNA; SEQ ID NO: 2 is nucleotide sequence of lower stand of DNA; SEQ ID NO: 3 is amino acid sequence of streptavidin protein.

FIG. 2 shows an SDS-PAGE analysis of the preparation of fusion protein. I: 20 μl of insoluble fraction; S: 20 μl of soluble fraction; L: Molecular weight marker; 1: 1 μl whole clarified extract post French press; 10: 10 μl whole clarified extract post French press; 20: 20 μl whole clarified extract post French press.

FIG. 3 shows an SDS PAGE analysis of the purified immunoglobulins using an affinity matrix according to the present invention. Loading pattern was M ; marker; 1: Bovine serum diluted 1+4; 2: Elute A (from streptavidin-protein A zz domain fusion protein beads prepared according to examples 1 and 2; 3: Elute B (from biotin beads only); 4: Elute C (from commercial protein A beads). Table 1 shows amounts loaded.

FIG. 4 is shows an SDS PAGE analysis of purification of IgG from bovine serum using an affinity matrix according to the present invention. Table 2 shows amounts loaded.

### MODE(S) FOR CARRYING OUT THE INVENTION

### Definitions

The term “affinity separation” as used herein refers to a method of separating, purifying, removing, enriching and/or concentrating a component from a mixture or suspension.

The term “fusion protein” as used herein means a protein having at least two elements, one element capable of binding strongly to a matrix and a second element capable of releasably binding a component.

The term “base matrix” as used herein means is any suitable matrix that can be used in a separation environment and can bind a fusion protein which further is capable of binding, or being bound by, a target component.

The term “matrix binding element” as used herein means is any suitable protein or peptide element that can bind to biotin.

The term “target binding element” as used herein means is any suitable element capable of binding a target in an affinity purification.

Biotin, also known as vitamin H or B₇, has the chemical formula C₁₀H₁₆N₂O₃S (Biotin; Coenzyme R, Biopeiderm), is a water-soluble B-complex vitamin which is composed of an ureido (tetrahydroimidizalone) ring fused with a tetrahydrothiophene ring. A valeric acid substituent is attached to one of the carbon atoms of the tetrahydrothiophene ring. The term “biotin” as used herein means biotin, biotin-like molecules or derivatives of biotin or modified forms of biotin that are capable of binding to avidin or streptavidin.

Native avidin is a 67-68 kDa tetramemeric protein found in the egg white of birds, reptiles, and amphibians. The term “avidin” as used herein includes native and recombinant monomeric as well as oligomeric forms of avidin. Use of the term “avidin” also contemplates fragments and variants of avidin that have the ability to bind strongly to biotin or derivatives of biotin.

Streptavidin is a 60 kDa tetrameric protein from the microorganism Streptomyces avidinii. The term “streptavidin” as used herein includes the native protein and recombinant monomeric as well as oligomeric forms thereof. Use of the term “streptavidin” also contemplates fragments and variants of streptavidin that have the ability to bind strongly to biotin or derivatives of biotin.

Protein A is a 40-60 kDa surface protein originally found in the cell wall of the bacteria Staphylococcus aureus. Protein A consists of five domains which each have the ability to bind to certain immunoglobulins. The term “protein A” as used herein includes the protein and any combination of the five immunoglobulin binding domains as well as any fragments or variants of any of the domains that are capable of binding an immunoglobulin.

The term “polypeptide” as used herein means a polymer made up of amino acids linked together by peptide bonds, and includes fragments or analogues thereof. The terms “polypeptide” and “protein” are used interchangeably herein, although for the purposes of the present invention a “polypeptide” may constitute a portion of a full length protein or a complete full length protein.

The term “nucleic acid” as used herein refers to a single- or double-stranded polymer of deoxyribonucleotide, ribonucleotide bases or known analogues of natural nucleotides, or mixtures thereof. The term includes reference to a specified sequence as well as to a sequence complimentary thereto, unless otherwise indicated. The terms “nucleic acid” and “polynucleotide” are used herein interchangeably.

The term “variant ” as used herein refers to substantially similar sequences. Generally, polypeptide sequence variant possesses qualitative biological activity in common. Further, these polypeptide sequence variants may share at least 50%, 55%, 60%, 65%, 70%, 75%, 80%, 85%, 90%, 95%, 96%, 97%, 98% or 99% sequence identity. Also included within the meaning of the term “variant” are homologues of polypeptides of the invention. A homologue is typically a polypeptide from a different species but sharing substantially the same biological function or activity as the corresponding polypeptide disclosed herein. Variant therefore can refer to a polypeptide which is produced from the nucleic acid encoding a polypeptide, but differs from the wild type polypeptide in that it is processed differently such that it has an altered amino acid sequence. For example a variant may be produced by an alternative splicing pattern of the primary RNA transcript to that which produces a wild type polypeptide.

The term “fragment” refers to a polypeptide molecule that encodes a constituent or is a constituent of a polypeptide of the invention or variant thereof. Typically the fragment possesses qualitative biological activity in common with the polypeptide of which it is a constituent. The term “fragment” therefore refers to a polypeptide molecule that is a constituent of a full-length polypeptide and possesses at least some qualitative biological activity in common with the full-length polypeptide. The fragment may be derived from the full-length polypeptide or alternatively may be synthesised by some other means, for example chemical synthesis.

The term “substantially” as used herein means the majority but not necessarily all, and thus in relation to a modified polypeptide “substantially” lacking a component region of a corresponding wild-type polypeptide, the modified polypeptide may retain a portion of that component region. For example, a modified polypeptide “substantially” lacking a component region of a corresponding wild-type polypeptide may retain approximately 50 percent or less of the sequence of the component region, although typically the component region is rendered structurally and/or functionally inactive by virtue of the proportion of the sequences of the region omitted.

### Affinity Matrix

The present invention is based on the surprising and unexpected finding that affinity matrices can be prepared by contacting a plain or derivatised base matrix with fusion proteins in which at least one part of the fusion protein has a strong affinity to the base matrix and at least one part of the fusion protein can bind, or be bound, to a target component thus providing for relatively inexpensive and reliable affinity separation. The particular instance of affinity separation exemplified herein is readily understood and appreciated by persons skilled in the art as representing a general method of affinity separation suitable for the separation, purification, removal, enrichment and/or concentration of any desired or undesired component.

Accordingly, the present invention relates to an affinity matrix separating at least one target component from a mixture. The affinity matrix comprises a base matrix and at least one fusion protein having at least one part capable of binding to the base matrix and at least one part able to bind the target component of interest. When a mixture or sample is contacted with the affinity matrix, the target component selectively binds to the target binding element of the fusion protein.

Thus, binding of the target component to the affinity matrix allows the target component to be separated from the mixture. The target molecules can, if desired, be separated from the affinity matrix by elution through methods well known to persons skilled in the art.

### Target Components

The target component may comprise a protein, a peptide, a polypeptide, an immunoglobulin, biotin, an inhibitor, a co-factor, a substrate, an enzyme, a receptor, a monosaccharide, an oligosaccharide, a polysaccharide, a glycoprotein, a lipid, a nucleic acid, a cell or fragment thereof, a cell extract, an organelle, a virus, a biological extract, a hormone, a serum protein, a milk protein, a milk-derived product, blood, serum, plasma, a fermentation product a macromolecule or any other molecule or any combination or fraction thereof. The biological extract may be derived from any plant, animal, microorganism or protista.

The target component may be a desired target component, such as an immunoglobulin from serum. However, the target component may also be undesired, such as a contaminant.

### Base Matrix

The base matrix suitable for use in then present invention may be a particulate matrix such as beads made from cellulose, agarose, dextrane, acrylamide, polysulfone, polyamide, silica, or other suitable materials or combinations thereof typically used as matrices for separation of biological macromolecules. Alternatively the base matrix may be a continuous matrix such as a membrane made from cellulose, acrylamide, polysulfone, polyamide, PVDF, PVC, polypropylene, polyester, silica or any other suitable materials or combinations thereof.

The base matrix is derivatised with biotin or derivatives thereof to allow the attachment of the fusion protein.

### Fusion Proteins

The present invention contemplates use of fusion proteins that have been modified to contain at least one biotin-binding element that binds to a selected matrix containing biotin and at least one target binding element that is capable to bind, or be bound by, a target component. Typically these fusion proteins are created by recombinant DNA technology where nucleotide fragments encoding the desired proteins, peptides or fragments thereof are joined together with or without an interspaced nucleotide fragment encoding a spacer or linker region. One part of the fusion protein may be streptavidin, avidin, or fragments thereof capable of binding to a base matrix derivatised with biotin or derivatives thereof at least one other part of the fusion protein may comprise an antibody binding domain such as protein A, protein G, protein L, or a single chain antibody, avidin, streptavidin, an enzyme, an inhibitor, an antigenic determinant, an epitope, a binding site, a lectin, a polyhistidine, an oligohistidine, a receptor, a hormone, a signalling molecule, a polypeptide with specific or group specific binding capabilities, or any combination thereof.

### Binding of Fusion Protein(s) to Base Matrix

The fusion protein may be attached to the base matrix by mixing the base matrix with a sample such as an extract, supernatant, secretion, or lysate containing the fusion protein. Alternatively the sample can be passed through a bed, a column, or a sheet of the base matrix. The biotin-binding element of the fusion protein with strong affinity for the base matrix will bind to the base matrix, thus providing an affinity matrix with at least one remaining element of the fusion protein being able to bind, or be bound by, a target component. Any undesired components of the sample containing the fusion protein can be washed away from the affinity matrix by the use of suitable solutions such as buffers, that typically do not cause the release of the fusion protein from the base matrix.

The base matrix may be derivatised with biotin and the biotin-binding element of the fusion protein with strong affinity for the base matrix may be streptavidin , avidin or variations thereof. The binding between biotin and avidin or streptavidin is one of the strongest non-covalent bonds known between to biological molecules, and the dissociation of the two molecules requires extreme conditions, which would cause most proteins to denature. The base matrix can be derivatised with biotin or derivatives thereof in a number of ways known to those skilled in the art. One such method includes the chemical attachment of a NHS-biotin derivative to a matrix containing primary amine groups. The reaction between the NHS-group and the primary amine can occur at neutral to alkaline pH and result in the formation of stable amide bonds between the biotin derivative and the base matrix.

A matrix containing primary amine groups can be provided by reacting a polysaccharide based matrix such as cellulose or agarose with an epoxide such as epichlorohydrin or 1,4 butanediol diglycidyl ether in a highly alkaline solution; this will result in epoxy-groups being covalently attached to the base matrix. These epoxy-groups can then subsequently be reacted with an amine compound such as 1,3-diaminopropane at alkaline pH, which will result in a base matrix with covalently attached primary amine groups.

Alternatively, a base matrix containing biotin can be provided by activating a base matrix with epoxy groups and subsequently letting the epoxy activated matrix react with biotin derivatives containing amino groups, sulfhydryl groups, or hydroxyl groups.

### Binding of Target Component(s) to Affinity Matrix

Target components may be bound to the affinity matrix by conventional methods such as those usually employed in affinity separations. These include:

a) packing the matrix in a column and passing the mixture containing the target component through the packed column;

b) adding the matrix to a conical vessel such as employed in fluid bed separations followed by passing the mixture through the affinity matrix in a manner that causes the affinity matrix to become fluidised;

c) mixing the affinity matrix with the mixture in a vessel and subsequently separating the affinity matrix containing the target component from the mixture by means of sedimentation, centrifugation, or filtration;

d) if the base matrix is in the form of a membrane, the mixture is passed through the pores of the membrane.

### Recovery of Target Component(s)

The target component may be recovered from the affinity matrix to which the target component is bound, and this recovery may involve at least one elution step. In this regard, the relevant eluant(s) may comprise a solution with compounds imparting high or low pH, high or low salt concentrations or compounds with competitive binding capacity. Such solutions can comprise inorganic or organic acids or salts thereof, chaotropic salts, or compounds with competitive binding capacity. For example, a buffer comprising glycine adjusted to a pH in the range of about 1.5 to 4. Other examples include buffers comprising citric, acetic, succinic, lactic, tartric, formic, propionic, boric or phosphoric acids or salts thereof. The eluant may also comprise a solution of one or more inorganic acids, for example hydrochloric acid, sulphuric acid and nitric acid, or salts thereof such as sodium chloride, potassium chloride, ammonium chloride, sodium sulphate, potassium sulphate or ammonium sulphate. The eluant may also comprise chaotropic compounds such as urea, guanidine, potassium iodide, sodium iodide, thiocyanates, detergents, hydrophobic molecules such as organic solvents, or any other molecule capable of weakening, breaking or disrupting molecular structures or bonds.

The eluant(s) used in the elution step(s) may have a pH in the range of about 1.0 to about 14.0. The eluants may have ionic strengths in the range from about 1×10⁻³ to about 25.

### Kits

The present invention also provides kits for separating, purifying, removing, enriching and/or concentrating a component from a mixture or suspension, wherein the kits facilitate the employment of the systems and methods of the invention. Typically, kits for carrying out a method of affinity separation contain at least a number of the reagents required to carry out the method. Typically, the kits of the invention will comprise one or more containers, containing for example, matrices, wash reagents, and/or other reagents capable of releasing a bound component from a polypeptide or fragment thereof.

In the context of the present invention, a compartmentalised kit includes any kit in which matrices and/or reagents are contained in separate containers, and may include small glass containers, plastic containers or strips of plastic or paper. Such containers may allow the efficient transfer of reagents from one compartment to another compartment whilst avoiding cross-contamination of the samples and reagents, and the addition of agents or solutions of each container from one compartment to another in a quantitative fashion. Such kits may also include a container which will accept a test sample, a container which contains the affinity matrices used in the assay and containers which contain wash reagents (such as phosphate buffered saline, Tris-buffers, and like).

Typically, a kit of the present invention will also include instructions for using the kit components to conduct the appropriate methods.

Methods and kits of the present invention find application in any circumstance in which it is desirable to purify any component from any mixture.

### Examples

### Example 1

### Preparation of a Fusion Protein

### Preparation of Plasmid Encoding a Streptavidin-zz Domain Fusion Protein

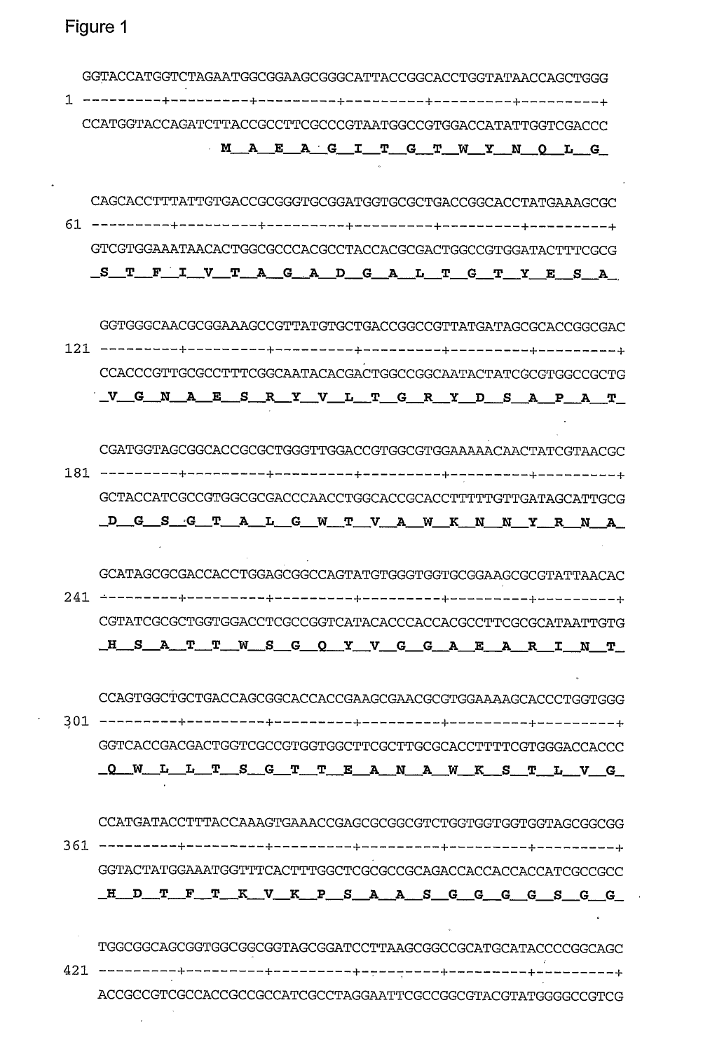

A gene construct containing a sequence encoding the biotin binding domain of Streptavidin followed by a sequence encoding a serine and glycine-rich linking region which in turn was followed by a sequence encoding the zz domain of protein A was prepared (FIG. 1). This sequence was PCR amplified and the NcoI-EcoRI inserts were subcloned into the pET-Duet1 vector. This vector was then transformed into the protease deficient E. coli strain BL21(DE3).

### Preparation of the Fusion Protein

An overnight culture of BL21 (DE3)-Tuner:pDuet-1:Sequence1 was grown in LB+Ap¹⁰⁰ at 37° C. This culture was used to seed three fresh cultures (25 ml overnight culture+500 ml LBA+Ap¹⁰⁰). The fresh cultures were grown for 2 hr at 37° C. IPTG was then added to a final concentration of 0.05 mM to induce protein expression. Cultures were then grown overnight (20.5 hr) at 22° C.

Solubility Check: Cells were pelleted from a 500 μl aliquot of overnight induction culture, resuspended in 100 μl BPER. The insoluble fraction was pelleted by centrifugation, the soluble supernatant fraction removed, and the insoluble pellet resuspended in 100 μl BPER.

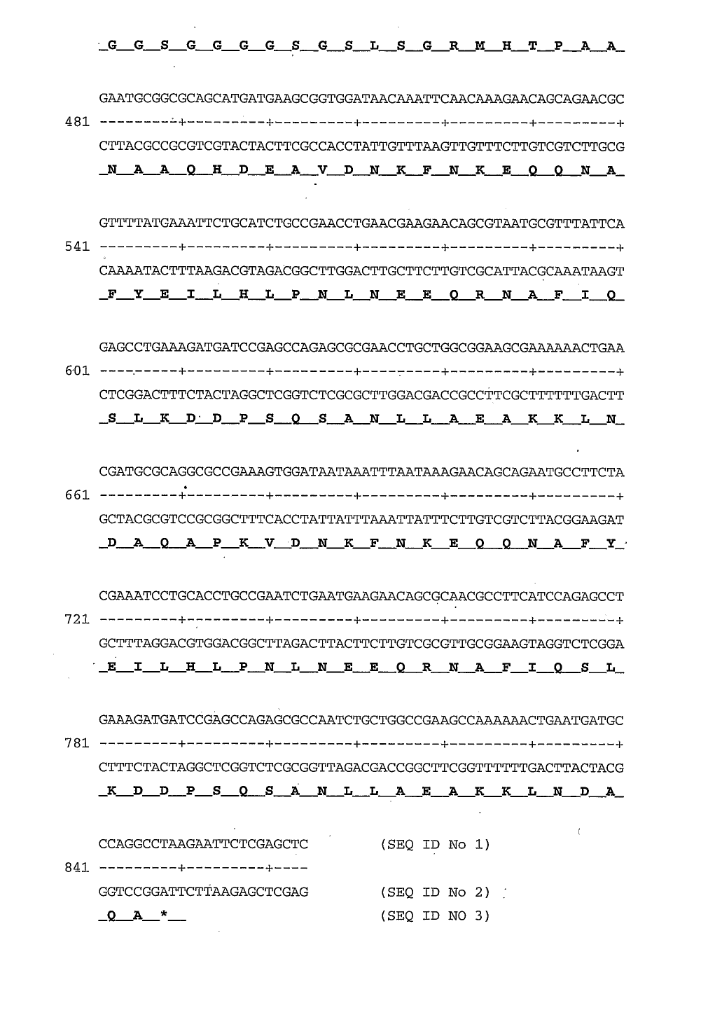

French Press: Cells were harvested from 1.5 l induction culture by centrifugation at 6000 rpm for 15 min: Cells were resuspended in 30 ml water. The cells were lysed by three passages through a French pressure cell. The cell lysates were clarified by centrifugation at 14000 rpm for 30 min (×2). Sodium azide was added to the clarified whole cell, lysate at a final concentration of 0.05%. Lysate was stored at 4° C. PAGE analysis of the various fractions is shown in FIG. 2.

### Example 2

### Preparation of an Affinity Matrix

100 μl of a 1:1 slurry of biotin agarose (Sigma B6885) in Tris buffered saline (TBS) was mixed with 1.5 ml of the cell lysate supernatant prepared as described in example 1. The streptavidin-Protein A zz-domain fusion protein was left to bind to the biotin-agarose for 30 min with occasional shaking. The agarose beads were left to settle and the beads were then washed 4 times with 1 ml TBS pH 7.4.

### Example 3

### Purification/Enrichment of Immunoglobulins from Serum

Three separate purification experiments were performed in parallel using the following matrices:
A) Streptavidin-Protein A zz domain fusion protein bound to biotin-agarose beads prepared as described in example 2
B) Biotin-agarose beads without any bound fusion protein (Sigma B6885)
C) Commercial protein A agarose (Sigma P3476)

100 μl of a 1:1 suspension of each bead type was placed in eppendorf tubes (i.e. 50 μl of settled gel volume), and 0.9 ml of bovine serum diluted 1+4 in TBS was added to each tube and left to bind for 30 min with regular shaking. The beads in all three tubes were then washed 4 times with 1 ml TBS pH 7.4.

Any bound protein was then eluted with 100 μl glycine pH 2.9. The supernatant (containing the eluted proteins) was then transferred to new tubes, and 10 μl 1M Tris pH 7.8 was then added to each elute.

### Analysis of Eluted Proteins:

12 μl of each sample was mixed with 4 μl loading buffer and the treated in a boiling water bath for 5 min.

### PAGE—Running of Gel:

Gel: NuPage 4-12% Bis-Tris gel (Invitrogen)

Running buffer: MES buffer with 500 μl “antioxidant” in upper buffer (Invitrogen)

Run time: 35 min @200V

Marker: Mark12 Unstained standard (Invitrogen)

Loading buffer: 1 ml NuPage LDS+50 μl β-mercaptoethanol

Staining: Coomassie Blue

### Loading of Gel:

TABLE 1

| | | Volume |
|---|---|---|
| Sample label | Description | loaded |
| M | Marker | 5 μl |
| 1 | Bovine serum diluted 1 + 4 | 5 μl |
| 2 | Elute A (from streptavidin-protein A zz domain | 10 μl |
| | fusion protein beads prepared according to | |
| | example 1 and 2 | |
| 3 | Elute B (from biotin beads only) | 10 μl |
| 4 | Elute C (from commercial protein A beads) | 10 μl |

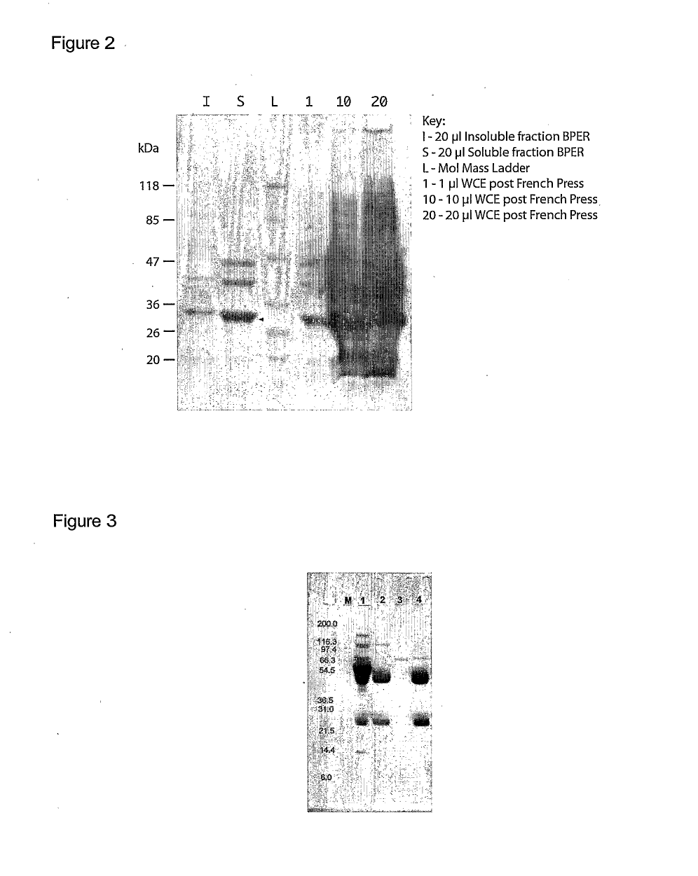

The gel is shown in FIG. 3.

The results show that heavy and light chains of immunoglobulins were purified from the bovine serum using the affinity matrix according to the present invention. The results furthermore show, that the affinity beads prepared according to the present invention are capable of purifying immunoglobulins from serum to approximately the same degree of purity as beads prepared by traditional methods.

### Example 4

### Preparation of Affinity Matrices

### Matrix

4% agarose beads activated with 1,4 butanediol diglycidyl ether approx. 40 μmol epoxy groups/ml beads

6% agarose beads activated with 1,4 butanediol diglycidyl ether approx. 70 μmol epoxy groups/ml beads

1 g of each agarose type was mixed with 100 μmol of each biotin reagent (4 reactions in total);

75 mg of Biotin-(PEO)₃-amine (MW 374.51) was dissolved in 1 ml 0.5M PO₄ buffer pH 12, and 0.5 ml was added to each of 1 ml of 4% and 6% activated agarose.

75 mg of Biotin-(PEO)₃-OH (MW 375.48) was first added to 2 ml 0.5M PO₄ buffer pH 13. 1 ml was then added to each of 1 ml of 4% and 6% activated agarose.

All four tubes were then rotated (on a slanting, rotating disc) for 24 hours at room temperature. The tubes were centrifuged, and the supernatant was taken off. 750 μl of 1M carbonate buffer pH 10.5 was added.

Excess epoxide groups were then blocked by adding 50 μl of ethanolamine to each microcentrifuge tube. The contents of the tubes was then allowed to react at room temperature for 4 hours before being washed with TBS pH 7.4 until pH reached ≦7.5.

A sample of 100 μl of a 1:1 suspension of each agarose derivative was pipetted into new tubes and washed with 1 ml of Tris buffered saline (TBS), followed by 1 ml 20 mM Tris pH 7.4, 1M NaCl. The supernatants were drawn off, and 1.2 ml of streptavidin-protein A cell supernatant from example 1 were added to each set of 50 μl beads respectively followed by 0.5 ml 1M Tris pH 7.8.

Each tube was incubated with cell supernatant for 1.5 hours while rotating on a slanting disc.

The matrices were then washed 1× with 20 mM Tris pH 7.2, 1 M NaCl, 1× with 20 mM Tris pH 7.8, 1×50 mM Glycine pH 1.9, and 1× with TBS (1.5 ml each).

### Example 5

### Purification of IgG from Bovine Serum and Comparison with Commercially Available Protein A Beads

### Matrix

4% and 6% agarose beads derivatised with biotin as described in example A and

GE Healthcare rProtein A Sepharose FF beads.

Samples of 100 μl of 1:1 suspensions of each agarose bead type were pipetted in to separate microcentrifuge tubes. 1.2 ml of bovine serum diluted 1+2 with Tris buffered saline (TBS) was then added to each tube, and the tubes were then incubated for 1 h on a rotating disc. The tubes were then washed 1× with TBS followed by 1× wash with 20 mM Tris, 1M NaCl pH 8, followed by 1×20 mM Tris pH 7.8 (all washes were with 1 ml). Bound IgG was then eluted with 200 μl 50 mM glycine pH 1.9. The supernatants (containing eluted protein) were transferred to fresh tubes containing 40 μl 1M Tris pH 7.8. All tubes were then re-eluted with 200 μl glycine pH 1.9 and the supernatant was pooled with the first elution. (i.e. total elution volume was 400 μl).

### SDS-Page

### Running of Gel:

Gel: NuPage 4-12% Bis-Tris gel (Invitrogen), 15 well.

Running buffer: MES buffer (with antioxidant in upper buffer chamber)

Run time: ˜40 min @200V

Marker: Mark12 Unstained standard (Invitrogen)

Loading buffer: NuPage LDS+5% β-mercaptoethanol

Staining: Coomassie Blue

All samples (apart from markers): 12 μl sample+4 μl loading buffer, followed by boiling for 5 min and then spun down in microcentrifuge tube.

Loading of gel is set out in Table 2.

TABLE 2

| Lane | Sample | Amount loaded (μl) |
|---|---|---|
| 1 | Marker | 8 |
| 2 | Bovine Serum diluted 1 + 4 | 5 |
| 3 | 4% NH₂ Biotin | 8 |
| 4 | 6% NH₂ Biotin | 8 |
| 5 | 4% OH Biotin | 8 |
| 6 | 6% OH Biotin | 8 |
| 7 | Marker | 8 |
| 8 | Bovine Serum diluted 1 + 4 | 5 |
| 9 | 4% NH₂ Biotin | 8 |
| 10 | 6% NH₂ Biotin | 8 |
| 11 | 4% OH Biotin | 8 |
| 12 | 6% OH Biotin | 8 |
| 13 | GE Healthcare rProtA | 5 |
| 14 | — | |
| 15 | Bovine Serum diluted 1 + 4 | 5 |

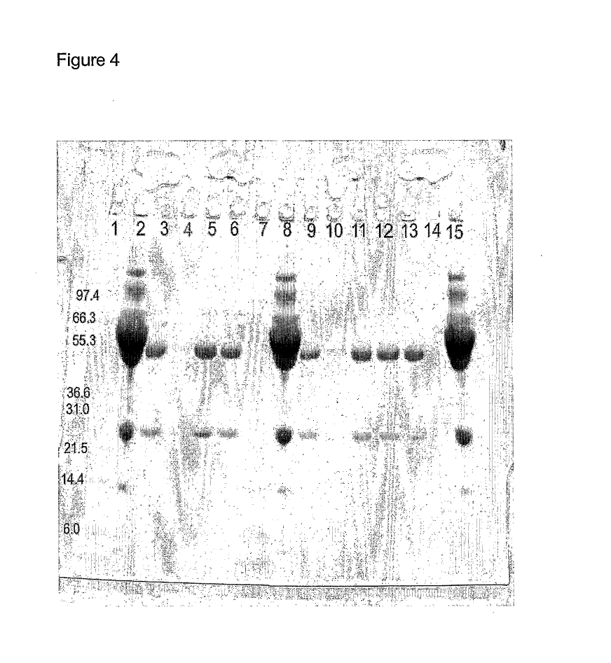

The results are shown in FIG. 4.

These results show that IgG with a purity and capacity equal to or higher than existing commercially available protein A matrices can be achieved by using an affinity matrix according to the present invention.

Protein A has been used as an example of a suitable target binding element of the fusion protein to demonstrate the present invention. It will be appreciated that other target binding elements can be used, depending on the target compound to be separated or purified.

### Example 6

### Multidomain Binding

The examples exemplify Streptavidin-Protein A and Protein A-streptavidin constructs. Other examples contemplated by the present invention include Streptavidin-streptavidin-Protein A, Streptavidin-Protein A-Streptavidin, Streptavidin-Protein A-Streptavidin-Protein A, etc. The rationale behind this is option is that multipoint attachment is generally much stronger than attachment through a single molecule.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

1.-22. (canceled)

23. An affinity matrix for binding a target component comprising:
a base matrix containing biotin; and
a fusion protein attached to the base matrix, wherein the fusion protein contains a matrix binding element capable of binding to the base matrix via biotin and a target binding element capable of binding, or being bound by, at least one target component.

24. The affinity matrix according to claim 23 wherein the base matrix is a particulate matrix comprising cellulose, agarose, dextrane, acrylamide, polysulfone, polyamide, silica, or combinations thereof.

25. The affinity matrix according to claim 23 wherein the base matrix is a continuous matrix comprising cellulose, acrylamide, polysulfone, polyamide, polyvinylidene difluoride, polyvinyl chloride, polypropylene, polyester, silica or combinations thereof.

26. The affinity matrix according to claim 23 wherein the base matrix is derivatised with biotin.

27. The affinity matrix according to claim 23 wherein the matrix binding element comprises streptavidin, or avidin.

28. The affinity matrix according to claim 27 wherein the matrix binding element comprises streptavidin.

29. The affinity matrix according to claim 23 wherein the target binding element is selected from the group consisting of protein A, protein G, protein L, an antibody binding domain, a single chain antibody, an enzyme, an inhibitor, an antigenic determinant, an epitope, a binding site, a lectin, a cellulose binding protein, a polyhistidine, an oligohistidine, a receptor, a hormone, a signalling molecule, a polypeptide with specific or group specific binding capabilities, and combinations thereof.

30. The affinity matrix according to claim 29 wherein the target binding element is protein A, protein G or protein L.

31. The affinity matrix according to claim 30 wherein the target binding element is protein A and the matrix binding element is streptavidin.

32. The affinity matrix according to claim 23 wherein the matrix includes a plurality of fusion proteins.

33. The affinity matrix according to claim 32 wherein the fusion proteins are capable of binding two or more different target components.

34. A method for separating at least one target component from a mixture comprising:
providing a mixture containing a target component to an affinity matrix comprising a base matrix containing biotin; and a fusion protein attached to the base matrix, wherein the fusion protein contains a matrix binding element capable of binding to the base matrix via biotin and a target binding element capable of binding, or being bound by, at least one target component; and
allowing a target component in the mixture to bind to the affinity matrix via the target binding element.

35. The method according to claim 34 further comprising:
recovering the target component from the affinity matrix.

36. The method according to claim 35 wherein at least one target component within a mixture is separated or removed from the mixture.

37. The method according to claim 34 wherein the mixture is selected from the group consisting of suspension, dispersion, solution and combination thereof of a biological source.

38. The method according to claim 37 wherein the biological source is selected from the group consisting of blood, blood plasma, blood serum, blood derived precipitates or supernatants, animal extracts or secretions, milk, colostrum, whey or any other milk derived product or fraction thereof, fermentation broths, liquids or fractions thereof, cell lysates, cell culture supernatants, cell extracts, cell suspensions, viral cultures or lysates, plant extracts and fractions thereof.

39. The method according to claim 34 wherein the target component is selected from the group consisting of a protein, a peptide, a polypeptide, an immunoglobulin, an inhibitor, a co-factor, a substrate, an enzyme, a receptor, a monosaccharide, an oligosaccharide, a polysaccharide, a glycoprotein, a lipid, a nucleic acid, a cell or fragment thereof, a cell extract, an organelle, a virus, a biological extract, a hormone, a serum protein, a milk protein, a milk-derived product, blood, serum, plasma, a fermentation product, a macromolecule or any other molecule and any combination or fraction thereof.

40. The method according to claim 39 wherein the target component is an immunoglobulin.

41. A method of making an affinity matrix comprising:
forming a base matrix by derivatising a matrix with biotin;
providing a fusion protein, wherein the fusion protein contains a matrix binding element capable of binding to the base matrix via biotin and a target binding element capable of binding, or being bound by, at least one target component; and
attaching the fusion protein to the base matrix via biotin.


SEQUENCE LISTING (3 sequences)

SEQ ID NO: 1 - DNA, 864 bp, Fungal
ggtaccatggtctagaatggcggaagcgggcattaccggcacctggtataaccagctgggcagcacctttattgtgaccgcgggtgcggatggtgcgctgaccggcacctatgaaagcgcggtgggcaacgcggaaagccgttatgtgctgaccggccgttatgatagcgcaccggcgaccgatggtagcggcaccgcgctgggttggaccgtggcgtggaaaaacaactatcgtaacgcgcatagcgcgaccacctggagcggccagtatgtgggtggtgcggaagcgcgtattaacacccagtggctgctgaccagcggcaccaccgaagcgaacgcgtggaaaagcaccctggtgggccatgatacctttaccaaagtgaaaccgagcgcggcgtctggtggtggtggtagcggcggtggcggcagcggtggcggcggtagcggatccttaagcggccgcatgcataccccggcagcgaatgcggcgcagcatgatgaagcggtggataacaaattcaacaaagaacagcagaacgcgttttatgaaattctgcatctgccgaacctgaacgaagaacagcgtaatgcgtttattcagagcctgaaagatgatccgagccagagcgcgaacctgctggcggaagcgaaaaaactgaacgatgcgcaggcgccgaaagtggataataaatttaataaagaacagcagaatgccttctacgaaatcctgcacctgccgaatctgaatgaagaacagcgcaacgccttcatccagagcctgaaagatgatccgagccagagcgccaatctgctggccgaagccaaaaaactgaatgatgcccaggcctaagaattctcgagctc

SEQ ID NO: 2 - DNA, 864 bp, Fungal
ccatggtaccagatcttaccgccttcgcccgtaatggccgtggaccatattggtcgacccgtcgtggaaataacactggcgcccacgcctaccacgcgactggccgtggatactttcgcgccacccgttgcgcctttcggcaatacacgactggccggcaatactatcgcgtggccgctggctaccatcgccgtggcgcgacccaacctggcaccgcacctttttgttgatagcattgcgcgtatcgcgctggtggacctcgccggtcatacacccaccacgccttcgcgcataattgtgggtcaccgacgactggtcgccgtggtggcttcgcttgcgcaccttttcgtgggaccacccggtactatggaaatggtttcactttggctcgcgccgcagaccaccaccaccatcgccgccaccgccgtcgccaccgccgccatcgcctaggaattcgccggcgtacgtatggggccgtcgcttacgccgcgtcgtactacttcgccacctattgtttaagttgtttcttgtcgtcttgcgcaaaatactttaagacgtagacggcttggacttgcttcttgtcgcattacgcaaataagtctcggactttctactaggctcggtctcgcgcttggacgaccgccttcgcttttttgacttgctacgcgtccgcggctttcacctattatttaaattatttcttgtcgtcttacggaagatgctttaggacgtggacggcttagacttacttcttgtcgcgttgcggaagtaggtctcggactttctactaggctcggtctcgcggttagacgaccggcttcggttttttgacttactacgggtccggattcttaagagctcgag

SEQ ID NO: 3 - protein, 277 aa, Fungal
MAEAGITGTWYNQLGSTFIVTAGADGALTGTYESAVGNAESRYVLTGRYDSAPATDGSGTALGWTVAWKNNYRNAHSATTWSGQYVGGAEARINTQWLLTSGTTEANAWKSTLVGHDTFTKVKPSAASGGGGSGGGGSGGGGSGSLSGRMHTPAANAAQHDEAVDNKFNKEQQNAFYEILHLPNLNEEQRNAFIQSLKDDPSQSANLLAEAKKLNDAQAPKVDNKFNKEQQNAFYEILHLPNLNEEQRNAFIQSLKDDPSQSANLLAEAKKLNDAQA